(19) United States
(12) Patent Application Publication
(10) Pub. No.: US 2022/0249416 A1
(43) Pub. Date: Aug. 11, 2022

(54) SYNERGISTIC COMBINATIONS OF SYNTHETIC LYSINE ANALOGS, DERIVATIVES, MIMETICS, OR PRODRUGS AND PHARMACEUTICAL AGENTS FOR ENHANCED EFFICACY

(71) Applicant: Tranexamic Technologies, LLC, Dallas, TX (US)

(72) Inventors: W. Paul Stewart, Dallas, TX (US); Frank Murdock, Dallas, TX (US)

(21) Appl. No.: 17/623,839

(22) PCT Filed: Oct. 29, 2020
(86) PCT No.: PCT/US2020/057877

§ 371 Date: Dec. 29, 2021

Related U.S. Application Data
(60) Provisional application No. 62/927,540, filed on Oct. 29, 2019.

(51) Int. Cl. A61K 31/195; A61K 31/197; A61K 31/454; A61K 31/522; A61K 31/7076; A61K 31/708; A61K 31/7068; A61K 31/506; A61K 31/215; A61K 31/045; A61K 31/52; A61K 31/7072

(52) U.S. Cl. CPC A61K 31/195; A61K 31/197; A61K 31/454; A61K 31/522; A61K 31/7076; A61K 31/7072; A61K 31/7068; A61K 31/506; A61K 31/215; A61K 31/045; A61K 31/52; A61K 31/708

(57) ABSTRACT

In an embodiment, the present disclosure pertains to a composition to enhance efficacy of a pharmaceutical agent. In some embodiments, the composition includes a synthetic lysine analog, derivative, mimetic, or prodrog and the pharmaceutical agent. In some embodiments, the synthetic lysine analog, derivative, mimetic, or prodrug and the antiviral agent form a synergistic combination. In an additional embodiment, the present disclosure pertains to a method to enhance efficacy of the pharmaceutical agent that generally includes administering the synergistic combination to a subject in need thereof. In a further embodiment, the present disclosure pertains to a kit to enhance efficacy of the pharmaceutical agent that generally includes the synergistic combination.

### CROSS-REFERENCE TO RELATED APPLICATIONS

This patent application claims priority from, and incorporates by reference the entire disclosure of, U.S. Provisional Application No. 62/927,540 filed on Oct. 29, 2019.

### TECHNICAL FIELD

The present disclosure relates generally to synergistic combinations and more particularly, but not by way of limitation, to compositions and methods for synergistic combinations of synthetic lysine analogs, derivatives, mimetics, or prodrugs and pharmaceutical agents for enhanced efficacy of the pharmaceutical agents, including, without limitation, synthetic chemical or biologically derived compounds, cells, other materials administered for medicinal purposes, and combinations thereof or for enhanced efficacy of the synthetic lysing analogs, derivatives, mimetics, or prodrugs.

### BACKGROUND

This section provides background information to facilitate a better understanding of the various aspects of the disclosure. It should be understood that the statements in this section of this document are to be read in this light, and not as admissions of prior art.

In various instances, two or more constituents that individually produce similar effects will sometimes display enhanced effects when given in combination. When a combined effect is greater than that predicted by individual potencies of each individual constituent, either by requiring lower concentrations or by reacting more positively at similar concentrations, the combination is said to be synergistic. A synergistic interaction can allow, for example, the use of lower concentrations of the combination constituents, a situation that can reduce adverse reactions of each individual constituent. As such, the present disclosure generally relates to synthetic lysine analogs, derivatives, mimetics, or prodrugs and pharmaceutical agents for enhanced efficacy of the pharmaceutical agents or the synthetic lysine analogs, derivatives, or mimetics based on synergistic effects.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a composition to enhance efficacy of a pharmaceutical agent. In some embodiments, the composition includes a synthetic lysine analog, derivative, mimetic, or prodrug and the pharmaceutical agent. In some embodiments, the synthetic lysine analog, derivative, mimetic, or prodrug and the antiviral agent form a synergistic combination.

In some embodiments, the synthetic lysine analog, derivative, mimetic, or prodrug can include, without limitation, tranexamic acid, epsilon-aminocaproic acid (EACA), and AZD 6564. In some embodiments, the synthetic lysine analog, derivative, mimetic, or prodrug is tranexamic acid. In some embodiments, the pharmaceutical agent can include, without limitation, a nucleoside analogue, a nucleobase analogue, a nucleotide analogue, antimicrobial agents, anticancer agents, genetic therapy agents, immune-enhancing agents, hormonal therapy agents, antiviral antibodies, and combinations thereof. In some embodiments, the pharmaceutical agent can include, without limitation, acyclovir, famciclovir, ganciclovir, penciclovir, valaciclovir, or valganciclovir, deoxyadenosine analogues, adenosine analogues, deoxycytidine analogues, guanosine and deoxyguanosine analogues, thymidine and deoxythymidine analogues, deoxyuridine analogues, didanosine, vidarabine, cytarabine, gemcitabine, emtricitabine, lamivudine, zalcitabine, abacavir, aciclovir, entecavir, stavudine, telbivudine, zidovudine, idoxuridine, trifluridine, oseltamivir, baloxavir marboxil, docosanol, and combinations thereof. In some embodiments, the pharmaceutical agent is acyclovir. In some embodiments, the pharmaceutical agent is docosanol. In some embodiments, the pharmaceutical agent is an adjuvant treatment or therapy agent. In some embodiments, the synergistic combination is in a solution. In some embodiments, the solution has a concentration of about 0.5 to about 30% by weight of the synthetic lysine analog, derivative, mimetic, or prodrug. In some embodiments, the solution is formulated as a spray, mist, aerosol, or mouthwash. In some embodiments, the solution is formulated to be applied as part of a vehicle which adapts to human skin. In some embodiments, the vehicle can include, without limitation, a gel, a lotion, and a cream. In some embodiments, the solution is formulated to be administered in a nasal passage. In some embodiments, the solution is formulated to be administered in an upper airway. In some embodiments, the solution is formulated to be administered intravenously. In some embodiments, the solution is formulated to be applied via a vehicle that allows the synergistic combination to be delivered in a time-released fashion. In some embodiments, the synergistic combination has a concentration of about 1% to about 60% by weight of the synthetic lysine analog, derivative, mimetic, or prodrug and about one-eighth up to about a standard dose or more of the pharmaceutical agent. In some embodiments, the synergistic combination is formulated to be delivered orally. In some embodiments, the synergistic combination is formulated to be delivered in a time-released fashion. In some embodiments, the synergistic combination treats or reduces occurrence of drug-resistant strains or mutations of a virus or other disease. In some embodiments, the synergistic combination is administered at least once per day.

In an additional embodiment, the present disclosure pertains to a method to enhance efficacy of a pharmaceutical agent. Generally, the method includes administering a synergistic combination to a subject in need thereof. In some embodiments, the synergistic combination includes a synthetic lysine analog, derivative, mimetic, or prodrug and the pharmaceutical agent.

In some embodiments, the synthetic lysine analog, derivative, mimetic, or prodrug can include, without limitation, tranexamic acid, epsilon-aminocaproic acid (EACA), and AZD 6564. In some embodiments, the synthetic lysine analog, derivative, mimetic, or prodrug is tranexamic acid. In some embodiments, the pharmaceutical agent can include, without limitation, a nucleoside analogue, a nucleobase analogue, a nucleotide analogue, antimicrobial agents, anticancer agents, genetic therapy agents, immune-enhancing agents, hormonal therapy agents, antiviral antibodies, and combinations thereof. In some embodiments, the pharmaceutical agent can include, without limitation, acyclovir, famciclovir, ganciclovir, penciclovir, valaciclovir, or valganciclovir, deoxyadenosine analogues, adenosine analogues, deoxycytidine analogues, guanosine and deoxyguanosine analogues, thymidine and deoxythymidine analogues, deoxyuridine analogues, didanosine, vidarabine, cytarabine, gemcitabine, emtricitabine, lamivudine, zalcitabine, abacavir, aciclovir, entecavir, stavudine, telbivudine, zidovudine, idoxuridine, trifluridine, oseltamivir, baloxavir marboxil, docosanol, and combinations thereof. In some embodiments, the pharmaceutical agent is acyclovir. In some embodiments, the pharmaceutical agent is docosanol. In some embodiments, the pharmaceutical agent is an adjuvant treatment or therapy agent. In some embodiments, the synergistic combination is in a solution. In some embodiments, the solution has a concentration of about 0.5 to about 30% by weight of the synthetic lysine analog, derivative, mimetic, or prodrug. In some embodiments, the solution is formulated as a spray, mist, aerosol, or mouthwash. In some embodiments, the solution is formulated to be applied as part of a vehicle which adapts to human skin. In some embodiments, the vehicle can include, without limitation, a gel, a lotion, and a cream. In some embodiments, the solution is formulated to be administered in a nasal passage. In some embodiments, the solution is formulated to be administered in an upper airway. In some embodiments, the solution is formulated to be administered intravenously. In some embodiments, the solution is formulated to be applied via a vehicle that allows the synergistic combination to be delivered in a time-released fashion. In some embodiments, the synergistic combination has a concentration of about 1% to about 60% by weight of the synthetic lysine analog, derivative, mimetic, or prodrug and about one-eighth up to about a standard dose or more of the pharmaceutical agent. In some embodiments, the synergistic combination is formulated to be delivered orally. In some embodiments, the synergistic combination is formulated to be delivered in a time-released fashion. In some embodiments, the synergistic combination treats or reduces occurrence of drug-resistant strains or mutations of a virus or other disease. In some embodiments, the administering is at least once per day.

In a further embodiment, the present disclosure pertains to a kit to enhance efficacy of a pharmaceutical agent. In some embodiments, the kit includes a synthetic lysine analog, derivative, mimetic, or prodrug and the pharmaceutical agent. In some embodiments, the synthetic lysine analog, derivative, mimetic, or prodrug and the antiviral agent form a synergistic combination.

In some embodiments, the synthetic lysine analog, derivative, mimetic, or prodrug can include, without limitation, tranexamic acid, epsilon-aminocaproic acid (EACA), and AZD 6564. In some embodiments, the synthetic lysine analog, derivative, mimetic, or prodrug is tranexamic acid. In some embodiments, the pharmaceutical agent can include, without limitation, a nucleoside analogue, a nucleobase analogue, a nucleotide analogue, an antimicrobial agent, an anticancer agent, a genetic therapy agent, an immune-enhancing agent, a hormonal therapy agent, an antiviral antibody, and combinations thereof. In some embodiments, the pharmaceutical agent can include, without limitation, acyclovir, famciclovir, ganciclovir, penciclovir, valaciclovir, or valganciclovir, deoxyadenosine analogues, adenosine analogues, deoxycytidine analogues, guanosine and deoxyguanosine analogues, thymidine and deoxythymidine analogues, deoxyuridine analogues, didanosine, vidarabine, cytarabine, gemcitabine, emtricitabine, lamivudine, zalcitabine, abacavir, aciclovir, entecavir, stavudine, telbivudine, zidovudine, idoxuridine, trifluridine, oseltamivir, baloxavir marboxil, docosanol, and combinations thereof. In some embodiments, the pharmaceutical agent is acyclovir. In some embodiments, the pharmaceutical agent is docosanol. In some embodiments, the pharmaceutical agent is an adjuvant treatment or therapy agent. In some embodiments, the synthetic lysine analog, derivative, mimetic, or prodrug is in a first medium and the pharmaceutical agent is in a second medium. In some embodiments, at least one of the first medium and the second medium is a pill, tablet, or capsule. In some embodiments, at least one of the first medium and the second medium is a solution. In some embodiments, the solution has a concentration of about 0.5 to about 30% by weight of the synthetic lysine analog, derivative, mimetic, or prodrug. In some embodiments, the solution is formulated to be administered in a nasal passage or an upper airway. In some embodiments, the solution is formulated to be applied as part of a vehicle which adapts to human skin. In some embodiments, the vehicle is selected from the group consisting of a gel, a lotion, and a cream. In some embodiments the synergistic combination has a concentration of about 1% to about 60% by weight of the synthetic lysine analog, derivative, mimetic, or prodrug and about one-eighth up to about a standard dose or more of the pharmaceutical agent. In some embodiments, the synergistic combination is administered at least once per day.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates antiviral activity of tranexamic acid (TA) and acyclovir (ACV), independently, at varying concentrations.

FIG. 2 illustrates effect of 2% TA on Herpes Simplex Virus Type 1 (HSV-1) DNA replication (multiplicity of infection (MOI) of 0.05).

FIG. 3 illustrates effect of 2% TA on HSV-1 DNA replication (MOI of 0.5).

FIG. 4 illustrates Herpes Simplex Virus (HSV) genes are transcribed in three temporal classes: (i) immediate early; (ii) early; and (iii) late.

FIG. 5 illustrates effect of 2% TA on infected cell protein 4 (ICP4) transcription (MOI of 0.5).

FIG. 6 illustrates effect of 2% TA on ICP4 transcription (MOI of 0.05).

FIG. 7 illustrates effect of 2% TA on infected cell protein 27 (ICP27) transcription (MOI of 0.5).

FIG. 8 illustrates effect of 2% TA on ICP27 transcription (MOI of 0.05).

FIG. 9 illustrates effect of 2% TA on infected cell protein 8 (ICP8) transcription (MOI of 0.5).

FIG. 10 illustrates effect of 2% TA on ICP8 transcription (MOI of 0.05).

FIG. 11 illustrates effect of 2% TA on thymidine kinase transcription (MOI of 0.5).

FIG. 12 illustrates effect of 2% TA on thymidine kinase transcription (MOI of 0.05).

FIG. 13 illustrates effect of 2% TA on glycoprotein C transcription (MOI of 0.5).

FIG. 14 illustrates effect of 2% TA on virion protein 16 (VP16) transcription (MOI of 0.5).

FIG. 15 illustrates effect of 2% TA on glycoprotein C transcription (MOI of 0.05).

FIG. 16 illustrates effect of 2% TA on VP16 transcription (MOI of 0.05).

FIG. 17 illustrates antiviral activity of TA and ACV, independently and in combination, at varying concentrations.

FIG. 18 illustrates TA dose-response (the half maximal inhibitory concentration (IC₅₀)=40.87 mM).

FIG. 19 illustrates antiviral activity (IC₅₀=40.87 mM).

FIG. 20 illustrates TA cytotoxicity of uninfected cells (cytotoxic concentration (CC₅₀)=320.3 mM TA).

FIG. 21 illustrates TA cytotoxicity of infected cells with an MOI of 0.5 normalized to Strain 17+ (CC₅₀=446.0 mM TA).

FIG. 22 illustrates effect of treatments on HO-1, a clinical isolate of HSV-1 that is multiple drug resistant, viral yields.

FIG. 23 illustrates a murine footpad HSV-1 latency model.

FIG. 24 illustrates that TA and ACV show synergy at reducing lethality of HSV-1 infection in the mouse footpad model.

FIG. 25 illustrates that tranexamic acid shows efficacy at reducing lethality of HSV-1 infection in the mouse footpad model.

FIG. 26 illustrates percent inhibition of HSV-1 yields with TA and docosanol, independently and in combination.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The three positively charged amino acids lysine, arginine, and histidine provide the positive charge in the electrostatic bond between positively and negatively charged residues required in multiple protein-protein, protein-DNA, and protein-RNA connections involved in various biological activities and the development of certain diseases, such as, for example, the replication of viruses. These three amino acids have similar chemical structures; thus, adding supplemental amounts of one or more can block the activity of one or more of the amino acids that are not supplemented. Similarly, providing a sufficient amount of an appropriate synthetic analog, derivative, or mimetic of lysine, arginine, or histidine can block the activity of one or more of the three amino acids.

In one aspect, the present disclosure generally relates to synthetic lysine analogs, derivatives, mimetics, and prodrugs (herein also referred to as “lysine analogs”), as they have been found to have multiple biological effects allowing the lysine analogs to be synergistically combined with other pharmaceutical agents, including, without limitation, synthetic chemical or biologically derived compounds, cells, other materials administered for medicinal purposes or therapies, and combinations thereof. Additionally, the converse is true; that is, the pharmaceutical agents can also synergistically enhance the effects of the lysine analogs. More specifically, but not by way of limitation, lysine analogs have an antifibrinolytic effect, an antiinflammatory effect, an antiviral effect, and an immune-enhancing effect, among other various biological effects. As such, a lysine analog can be beneficially combined with any pharmaceutical agent if it does not interfere with the mode of action of the pharmaceutical agent. Additionally, one or more of the biological effects of the lysine analog enhances the effects of the pharmaceutical agent by, for example, making its activity more rapid or complete, by allowing a lower dose of the pharmaceutical agent to be used, or by improving the condition or healing of a patient through secondary effects caused by the lysine analog. In addition, for diseases or conditions where genetic mutation may occur that reduces or eliminates the effectiveness of the pharmaceutical agent, the complementary activity of the lysine analog makes it more difficult for genetic mutation to avoid the combined effect of the pharmaceutical agent and the lysine analog, as multiple mutations would have to occur almost simultaneously. It should be understood that all synergistic benefits imparted onto the pharmaceutical agent can also be realized by the lysine analog imparted by the pharmaceutical agent.

As an example, Herpes Simplex Virus Type 1 (HSV-1) is a very common virus in the human population with various estimates indicating that up to 80% of the world's population is a carrier of HSV-1, and around 30% have recurrent cold sore outbreaks due to HSV-1. One approach to treatment is the use of antiviral medications, such as, for example, acyclovir or docosanol (behenyl alcohol). As described in more detail below, tranexamic acid (a lysine analog) has also been shown to inhibit HSV-1, and that its mode of action against HSV-1 is different from that of acyclovir. As such, the two can act synergistically to inhibit HSV-1 through two different modes of action, and additionally, a lower dose of each can be used in a synergistic combination while achieving similar effects against viral replication. Furthermore, using the synergistic combination of agents with different methods of action reduces the ability of the virus to develop resistance. In addition, if the strain of the virus that is being treated (e.g., HSV-1) happens to be one of the strains that are already resistant to the antiviral medication (e.g., acyclovir) a synergistic combination that includes tranexamic acid will still be effective. Moreover, because tranexamic acid itself affects multiple aspects of the replication of HSV-1, tranexamic acid provides an additional degree of effectiveness and avoidance of viral resistance. Additionally, tranexamic acid's antifibrinolytic effect, antiinflammatory effect, and immune enhancement effects provide secondary benefits for a more rapid healing of blisters and other aspects of a cold sore outbreak, when the particular virus of interest is HSV-1.

Antivirals, such as, but not limited to, acyclovir, are used to decrease pain and speed the healing of, for example, sores or blisters in people who have varicella (chickenpox), herpes zoster (shingles), first-time or repeat outbreaks of HSV-1 or Herpes Simplex Virus Type 2 (HSV-2), or various other viral infections. Additionally, docosanol is an example of a cold sore medication that penetrates the skin and blocks the virus while additionally providing a barrier for healthy cells. Antivirals are additionally sometimes used prophylactically to prevent or suppress outbreaks of sores or blisters in people who are infected with HSV-1, HSV-2, or other types of recurrent viral outbreaks or dormant viral infections. Some antivirals, such as acyclovir, are in a class of antiviral medications known as nucleoside analogues. As acyclovir is a nucleoside analogue, it is envisioned that any nucleoside or nucleoside analogue antivirals with a similar mechanism of action, for example, reliance on thymidine kinase as discussed in detail below, would interact with lysine analogs in a similar fashion to that of acyclovir. As further disclosed below, tranexamic acid does not affect gene transcription of, for example, thymidine kinase, and as such, tranexamic acid can be utilized synergistically with classes of drugs that rely on similar mechanisms of action for multiple antiviral purposes. In addition, it is further envisioned that synergistic benefits can be realized with any antiviral medication such as, for example, remdesivir that has a method of action differing from the method of action of lysine and lysine analogs. This synergistic benefit is also realized in natural and recombinant antibody treatments in which the method of actions are unrelated to that of the method of action for lysine and lysine analogs.

Nucleoside analogues are highly potent and selective inhibitors of viral enzyme thymidine kinase. Nucleoside analogues depend on the activity of the viral thymidine kinase to convert the analogue to a monophosphate form and subsequently interfere with viral DNA replication. The antiviral activity of nucleoside analogues relies on the fact that viruses encode their own nucleoside kinases having much lower substrate specificity than their cellular counterparts. Therefore, they are able to monophosphorylate certain nucleoside analogues whereas cellular nucleoside kinases cannot do so, or only to a very limited extent. The resulting analogue monophosphates are metabolized, by cellular kinases, to the respective triphosphates, which show distinctly lower molar inhibitory constants for virus-encoded DNA polymerases than for cellular DNA polymerases. This step can selectivity causes obligate chain termination, thus resulting in the cessation of viral production.

As an illustrative example, and not by way of limitation, acyclovir undergoes monophosphorylation catalyzed by a virus-encoded enzyme thymidine kinase. The formation of the monophosphate can only take place in the presence of the virus, thus acyclovir accumulates as the monophosphate only in infected cells. It is then converted to a diphosphate and triphosphate by normal host enzymes in the cell. This in turn inhibits the viral DNA polymerase from incorporating guanosine triphosphate, and is itself incorporated. The DNA cannot grow further and the chain terminates.

The mechanism of action is threefold: (i) competitive inhibition of viral DNA polymerase; (ii) chain termination of DNA once it has been incorporated into the nucleic acid; and (iii) inactivation of the viral DNA polymerase acid. This class of antiviral agents can be used against, for example, the hepatitis B virus, the hepatitis C virus, the herpes simplex virus (HSV-1 and HSV-2), and the human immunodeficiency virus (HIV).

Lysine analogs have been shown to inhibit the replication of various viruses such as, for example, HSV-1, HSV-2, HIV, influenza A, influenza B, and the like, by antagonizing one or more of lysine, arginine, and histidine. With respect to HSV-1, an illustrated example demonstrated herein, the lysine analog tranexamic acid inhibits viral replication by interfering with the transcription of at least four different genes. In addition, tranexamic acid inhibits the replication of HSV-1 likely through the same mechanisms as natural lysine. Various studies have been conducted to verify that arginine supports viral growth of HSV-1 and that lysine antagonizes this action of arginine. The action of lysine is multifactorial with one mechanism involving the histone layer around the DNA of the host eukaryotic cell. Five different types of histones have been identified and are synthesized only during DNA replication, where lysine-rich histones crosslink DNA fibrils of chromatin during metaphase and interphase, making the chromatin more compact and thus maintaining the structural integrity of the human chromosome. The DNA nucleoside compositions of viruses contain a higher ratio of arginine to lysine, and the infected cell synthesizes proteins of higher arginine to lysine ratio. Viruses, such as herpes simplex, make frequent use of the guanine(G)-containing codons, whereas human host cells have infrequent use of the cytosine(C)-guanine. There are six arginine codons and only two lysine codons. A simple shift of one nucleotide produces arginine. This can occur quite rapidly in the translation apparatus of an infected host cell. Lysine-rich host-cell proteins are altered by the viral DNA, and new arginyl tRNA synthesizing arginine-rich proteins are produced.

Lysine and lysine analogs also antagonize arginine, with respect to HSV-1, for example, by appearing to be an antimetabolite and analog of arginine, competing for reabsorption at the renal tubules, resulting in increased arginine excretion, competing for transport across the intestinal wall, acting as an arginase inducer, resulting in degradation of arginine, and decreasing the intracellular content of arginine in the tissue cells by entering the transport system. In addition, for viruses that replicate in the cell nucleus, a nuclear localization sequence (NLS) is required for every protein that the virus creates in the cytoplasm of the cell that must enter the nucleus for formation of new virions, and every NLS includes a series of lysine and arginine residues on the protein. Moreover, by the blocking of lysine and arginine residues lysine analogs can alter the extra cellular environment to inhibit viral attachment and entry in additional to the intra cellular actions discussed in detail below.

Described herein, are illustrative examples showing that tranexamic acid also inhibits transcription of certain genes required for the replication of HSV-1. As shown below, tranexamic acid inhibits the transcription of at least two immediate early (JE) stage genes, and possibly the transcription of at least two late stage genes, but it does not inhibit significantly the transcription of the early stage gene for thymidine kinase, which is the enzyme needed by acyclovir, or other nucleoside analogue antiviral agents, to start its process to become acyclovir triphosphate, which blocks replication of viral DNA. Accordingly, the present disclosure provides an example of the synergistic combination of a lysine analog, tranexamic acid, with a pharmaceutical agent, such as acyclovir, to provide enhanced efficacy of the pharmaceutical agent, namely to inhibit viral replication of HSV-1. Based on data presented herein, other nucleoside analogue antiviral agents are readily envisioned that have similar methods of action as acyclovir. Furthermore, the benefits of lysine analogs, such as tranexamic acid, also benefit from a synergistic combination.

Individual antiviral activity of tranexamic acid and acyclovir is shown in FIG. 1, which illustrates the antiviral activity of tranexamic acid and acyclovir, independently, at varying concentrations. As briefly discussed above, two or more compounds that individually produce similar effects can sometimes display enhanced effects when given in combination. When a combined effect is greater than that predicted by individual potencies of each individual constituent, for example, either by requiring lower concentrations or by reacting more positively at similar concentrations, the combination is said to be a synergistic combination. This synergistic interaction allows, for example, the use of lower concentrations of the combination constituents, a situation that can reduce adverse reactions of each individual constituent. In addition, secondary effects of one of the combination constituents that enhance the primary effect, or provide other benefits such as faster healing, may also be considered synergistic. For example, lysine analogs inhibit the activation of plasminogen into plasmin. Plasmin breaks down fibrin clots, has inflammatory effects, and has negative effects on certain immune functions. Additionally, plasmin can affect virulence in, for example, influenza viruses, such that limiting plasmin results in antiviral effects. Therefore, by inhibiting the formation of plasmin, among other mechanisms, lysine analogues are effective antifibrinolytic, anti-inflammatory, and immune-enhancing agents.

Accordingly, the present disclosure seeks to harness synergistic combinations of pharmaceutical agents, such as acyclovir, and lysine analogs, such as tranexamic acid, in order to enhance the efficacy of the pharmaceutical agent and the lysine analog. This can be done by, for example, using varying doses of each of the pharmaceutical agent and the lysine analogs to achieve an equivalent effect, or relying on the synergistic combination utilizing the same dosing as would be administered individually, yielding a higher effect. For example, the lysine analogs could be in a higher or lower concentration as compared to the pharmaceutical agent, or the pharmaceutical agent could be in a higher or lower concentration as compared to the lysine analogs.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

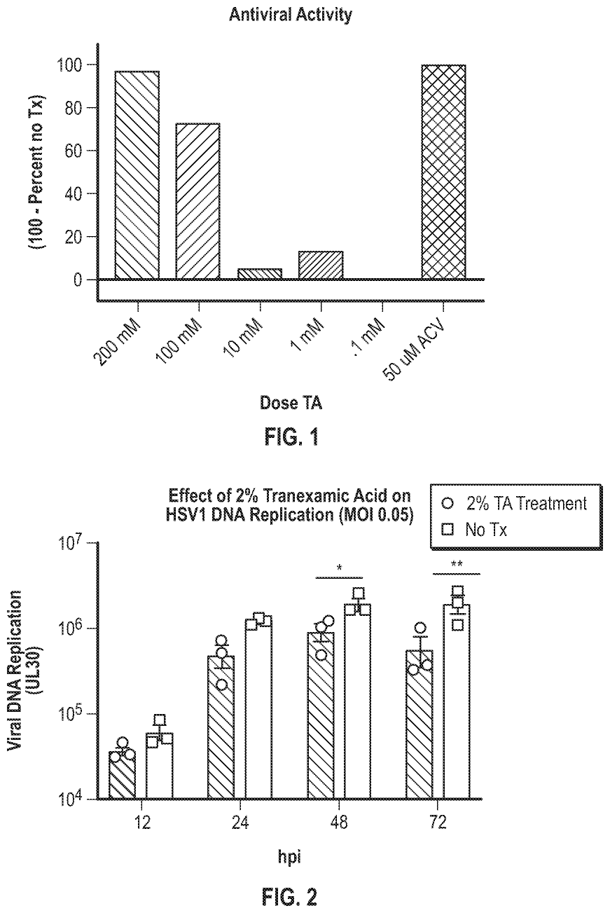

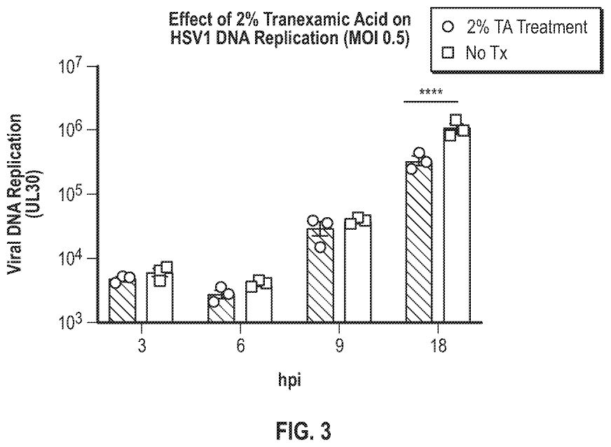

Effect of 2% Tranexamic Acid on HSV-1 Transcription. It has been previously demonstrated that tranexamic acid significantly reduces the production of infectious viruses during high and low multiplicity HSV infections and that tranexamic acid significantly reduces the replication of HSV DNA, as shown in FIG. 2 and FIG. 3. As such, it was sought to determine if tranexamic acid interferes with the expression of HSV genes during infection.

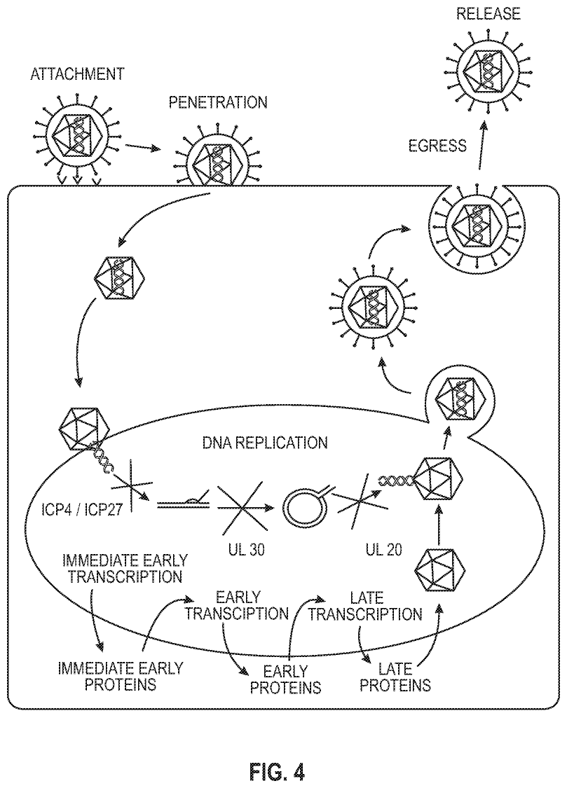

Examining the Effect of Tranexamic Acid on HSV Transcription. HSV genes are transcribed in three temporal classes (FIG. 4): (i) immediate early (IE)-transcribed immediately after infection; (ii) early (E)-transcribed 2 to 4 h post infection; and (iii) late (L)-transcribed after DNA replication. The present disclosure utilized real-time polymerase chain reaction (RT-qPCR) to examine effects of tranexamic acid on the transcription of select genes of each of the three classes. In this way the present disclosure assesses at what time point(s) after infection tranexamic acid was interfering with infection.

Tranexamic Acid's Effect on HSV Transcription. Rabbit skin cell monolayers were infected with HSV-1 Strain 17+ at a multiplicity of infection (MOI) of 0.5 or 0.05. Half of a 24-well plate of rabbit skin cells was treated with 2% (127.2 mM) tranexamic acid and the other half with vehicle. The plate was then incubated for 2 h at 37° C., 5% CO₂. All wells were infected with HSV Strain 17+ at an MOI of 0.5 or 0.05, and the virus was allowed to adsorb to the cells for 1 h. After the 1 h incubation, inoculum was removed and replaced with media containing either 2% tranexamic acid or vehicle, and incubated at 37° C., 5% CO₂. For the MOI of 0.5 series wells were harvested wells at 3, 6, 9, and 18 hpi. For the MOI of 0.05 series wells were harvested at 12, 24, 48, and 72 hpi. RNA was extracted and purified from the cells and treated with deoxyribonuclease (DNase) and reverse transcription to produce cDNA for quantitative polymerase chain reaction (qPCR) analysis was performed. qPCR on cDNA was performed from each treatment group and at time point probing for representative HSV IE, E, and L genes.

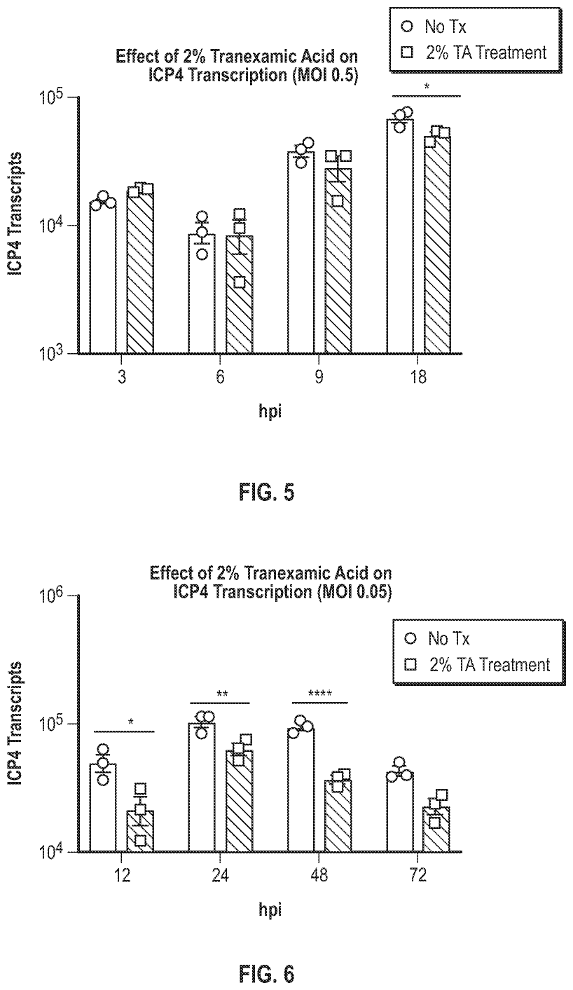

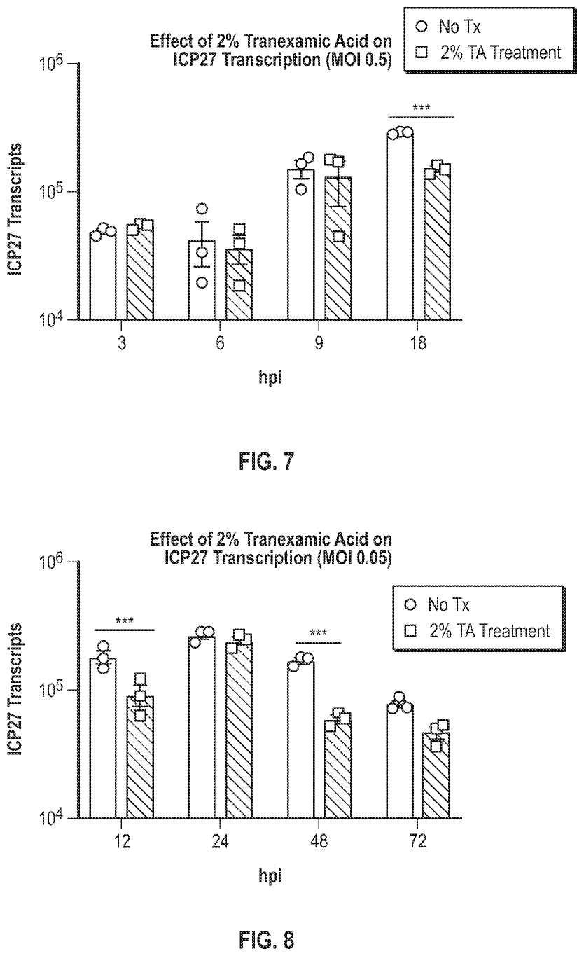

Effect of 2% Tranexamic Acid on the Accumulation of HSV Immediate Early Gene RNA (MOI of 0.5 and 0.05). FIG. 5 though FIG. 8 illustrate tranexamic acid on infected cell protein 4 (ICP4) (viral transactivator) and infected cell protein 27 (ICP27) (regulator of splicing, RNS export from nucleus). These viral genes are expressed immediately after entry of the viral DNA into the nucleus, and their expression is used in early gene expression. In conclusion, there is a modest, though significant, decrease of 2- to 4-fold of both ICP27 and ICP4 RNA suggesting that tranexamic acid acts very early after infection in interfering with IE gene transcription/accumulation. Note that the effect is greater at lower MOI (0.05).

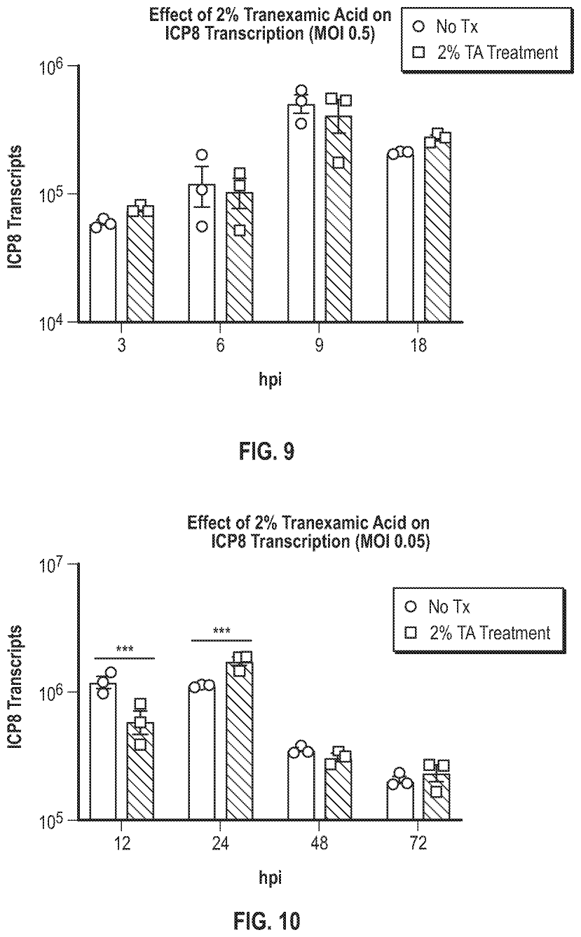

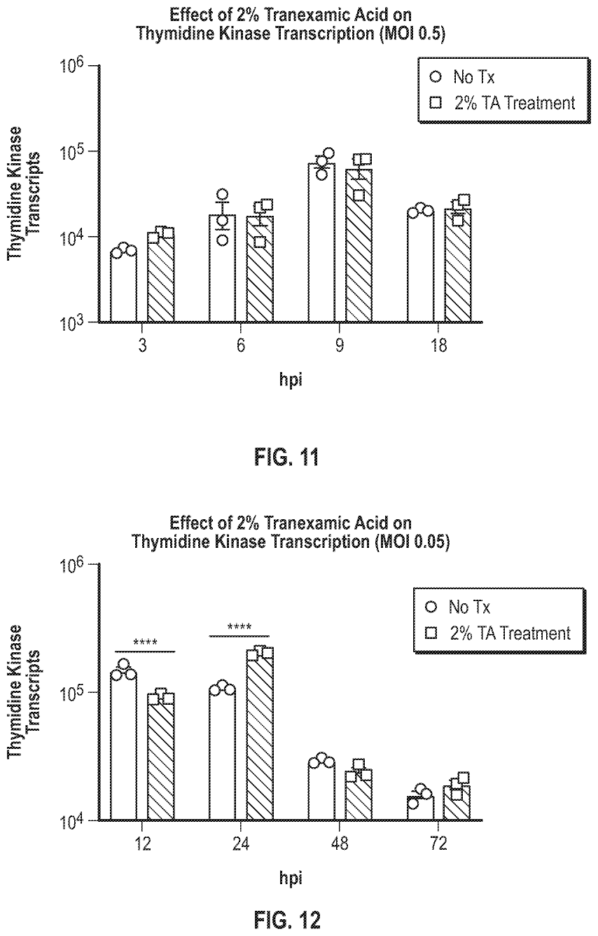

Effect of 2% Tranexamic Acid on the Accumulation of HSV Early Gene RNA (MOI of 0.5 and 0.05). FIG. 9 through FIG. 12 illustrate tranexamic acid on infected cell protein 8 (ICP8) (HSV DNA binding protein, a precursor for HSV DNA replication) and thymidine kinase (kinase for increasing nucleotide pools for HSV DNA replication). These viral genes are expressed after IE genes are made and activate their transcription. The early genes, as a group, make proteins that are used in HSV DNA replication.

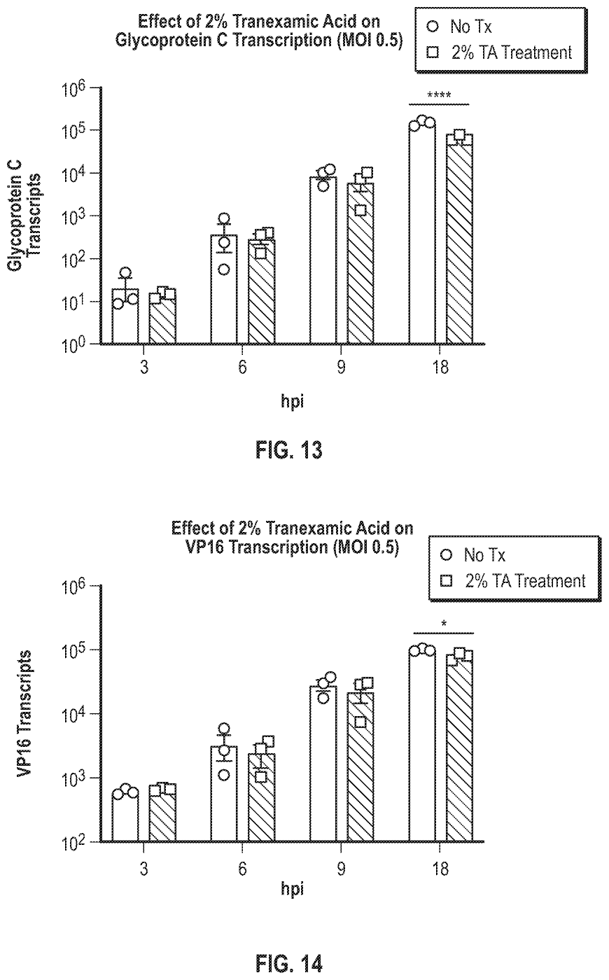

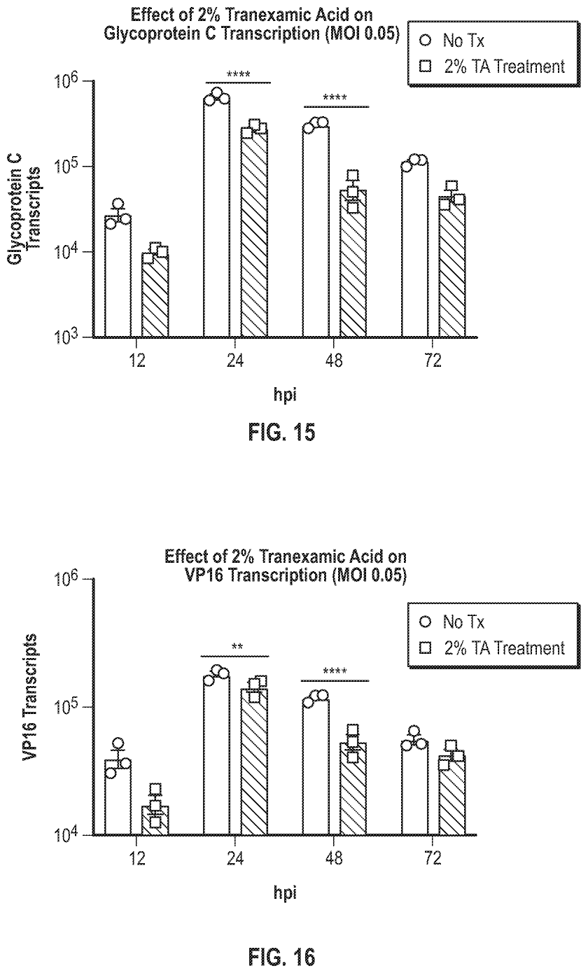

Effect of 2% Tranexamic Acid on the Accumulation of HSV Late Gene RNA (MOI of 0.5 and 0.05). FIG. 13 through FIG. 16 illustrate tranexamic acid on glycoprotein C (gC), a virion envelope component that is used for attachment of HSV to cells and virion protein 16 (VP16), a component of the virion “tegument” that is used in transactivating viral IE genes. These viral genes are expressed after viral DNA replication has occurred. The late genes, as a group, make proteins that are structural proteins used to produce the virus particles (virions).

In conclusion, the results above indicate that tranexamic acid significantly reduces the accumulation of late viral RNA at both high and low MOI. As such, it has been shown that tranexamic acid significantly reduces IE gene expression by 2- to 4-fold, and the effect is greater at lower MOI. In contrast, tranexamic acid generally does not cause a significant decrease in early gene transcription. This suggests that tranexamic acid may be affecting HSV transcription in a promoter/transcription factor-dependent manner. In addition, tranexamic acid significantly reduces late gene expression (approximately up to 8-fold) at both high and low MOI. Without being bound by theory, this may be the result of specific effects of tranexamic acid on initiation of transcription from HSV late promoters or a consequence of its effects on viral DNA replication. This data points to a novel mechanism of blocking viral transcription that occurs very early after infection, a property that may have significant therapeutic advantages, especially when combined with antiviral agents operating under different mechanisms, as discussed in further detail below.

Tranexamic Acid and Acyclovir. Rabbit skin cell monolayers were infected with HSV-1 Strain 17+ and treated with either tranexamic acid or acyclovir alone, or with tranexamic acid and acyclovir in combination forming a synergistic combination. Wells of a 24-well plate of rabbit skin cells were pretreated with different combinations of doses of tranexamic acid and acyclovir, alone and in combination, for 2 h. Wells were infected with HSV-1 Strain 17+ at an MOI of 0.05 and absorbed for 1 h. Infected monolayers were overlain with media containing appropriate concentrations of tranexamic acid or acyclovir alone, or with tranexamic acid and acyclovir in combination. Wells were then harvested at 24 hpi and DNA purification and quantitative polymerase chain reaction (qPCR; TagMan) probing for the HSV-1 UL30 gene region was performed. All treatments were performed in triplicate and one complete replicate of the experiment was performed.

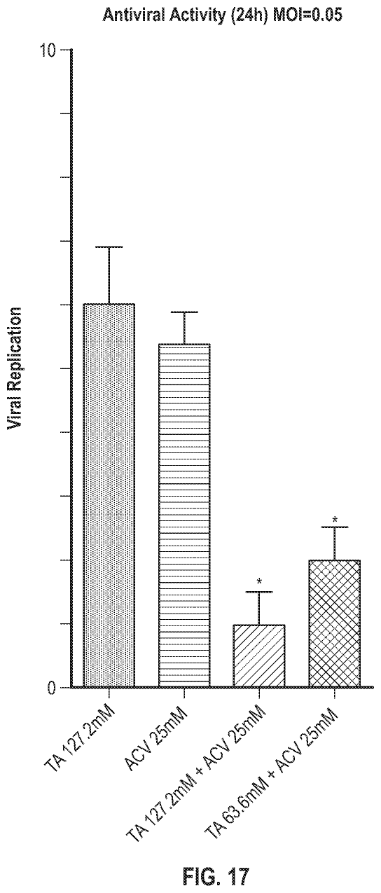

As illustrated in FIG. 17, experimental data indicated that use of 127.2 mM tranexamic acid in combination with 25 uM acyclovir suppressed HSV-1 replication approximately 5-fold more than either tranexamic acid or acyclovir alone. In addition, experimental data indicated that using half the dose of tranexamic acid in combination with acyclovir was synergistically effective at reducing HSV-1 replication approximately 4-fold.

As demonstrated above, tranexamic acid, in combination with acyclovir, acts synergistically to reduce HSV-1 replication in vitro. Furthermore, even when each of tranexamic acid and acyclovir are used at sub-effective dose 90 (sub-ED90), the synergism of the combination results in a 4- to 5-fold enhancement in antiviral suppression when compared to either compound individually. Without being bound by theory, it is believed that the two compounds act by inhibiting different components of the HSV-1 infection program, which likely contributes to this synergistic effect.

Tranexamic Acid Inhibits HSV-1 Virus Production In Vitro: Dose and Toxicity Analysis. Confluent rabbit skin cell monolayers were infected with HSV-1 Strain 17syn+ at an MOI of 5. The comparative control was 50 μM acyclovir (ACV) and negative controls were mock (uninfected) and Strain 17syn+ infected (no drug treatment).

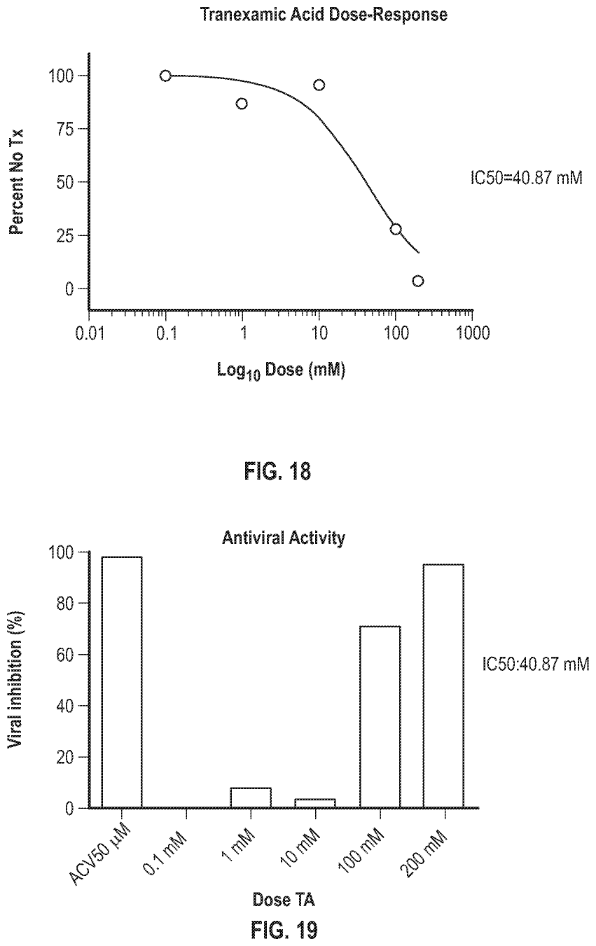

24-well plate of rabbit skin cells were pretreated in triplicate with appropriate treatment (mock, Strain 17+, 200 mM tranexamic acid (TA), 100 mM TA, 10 mM TA, 1 mM TA, 0.1 mM TA, 50 μM ACV) for 2 hrs. Cells were then infected with HSV-1 17syn+ at and MOI of 5 and the virus was allowed to adsorb for 1 hr. Cells and supernatant were harvested at 24 hr post infection. Plaque assays were performed on 10⁻¹ through 10⁻⁴ dilutions of samples to determine viral yields. The half maximal inhibitory concentration (IC₅₀) was then determined. FIG. 18 illustrates tranexamic acid dose-response (IC₅₀=40.87 mM). FIG. 19 illustrates antiviral activity (IC₅₀=40.87 mM).

Cytotoxicity Assay—50% Cytotoxic Concentration (CC₅₀). The cytotoxicity assay was performed in two arms: (1) uninfected cells without drug; and (2) cells infected with HSV-1 at an MOI of 0.5, with and without drug. MOI was 0.5 for HSV-1 Strain 17+ (for infected wells). The positive control was 50 μM ACV and the negative control was mock, Strain 17+ (no treatment). Blanks were no cells, only media. 96-well plate of rabbit skin cells were pretreated in triplicate (×2) with appropriate treatment (mock, Strain 17+, 200 mM TA, 100 mM TA, 10 mM TA, 1 mM TA, 0.1 mM TA, 50 uM ACV) for 2 hrs.

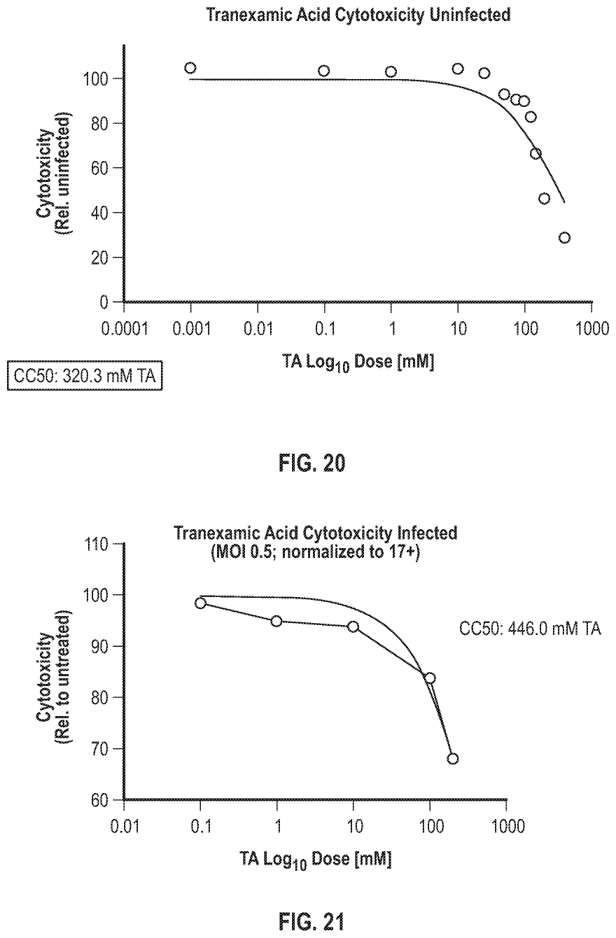

Half of the plate was infected with Strain 17+ at an MOI of 0.5 for 1 hr while the other half was left uninfected. Cells were post-treated for 48 hrs. CELLTITER-GLO® cytotoxicity assay was performed and CC₅₀ determined. FIG. 20 illustrates tranexamic acid cytotoxicity of uninfected cells (CC₅₀=320.3 mM TA). FIG. 21 illustrates tranexamic acid cytotoxicity of infected cells with an MOI of 0.5 normalized to Strain 17+ (CC₅₀=446.0 mM TA).

In conclusion, in the rabbit skin cell system, tranexamic acid has an ID₅₀ of 40.87 mM. At a dose of 100 mM the inhibition of HSV-1 is nearly 80% (close to that of acyclovir). The CC₅₀ is 320.3 mM for uninfected cells. Interestingly, the CC₅₀ for infected cells is greater (over 400 mM) and this is possibly due to HSV's anti-intrinsic response machinery. This data indicates that tranexamic acid has an ID₅₀ against HSV-1 in vitro that is well below its CC₅₀.

Effectivity of Tranexamic Acid against Acyclovir-Resistant PAAr5 Virus. While acyclovir is more effective than tranexamic acid against Strain 17+ there is the possibility that tranexamic acid acts as an antiviral against acyclovir-resistant strains of HSV-1. Acyclovir resistance develops by mutations in the HSV thymidine kinase and/or polymerase genes. Initial data strongly suggest that tranexamic acid acts by different mechanism(s) than acyclovir.

24-well plate of rabbit skin cells were pretreated, in triplicate, with 2% tranexamic acid and 50 uM acyclovir for 2 hrs. 6 wells utilized only media, 3 wells utilized mock, and 3 cells were utilized for no Tx control. All walls (except mock) were infected with HO-1, a clinical isolate of HSV-1 that is multiple drug resistant, at an MOI of 5 for 1 hr. Wells were post-treated in the same manner as pretreatment. At 24 hr hpi, each well was harvested in a micro-centrifuge tube. Freeze-that cycles were performed for each sample. Plaque assay for 10⁻¹ thru 10⁴ dilutions of each sample were performed and average titer for each treatment was determined.

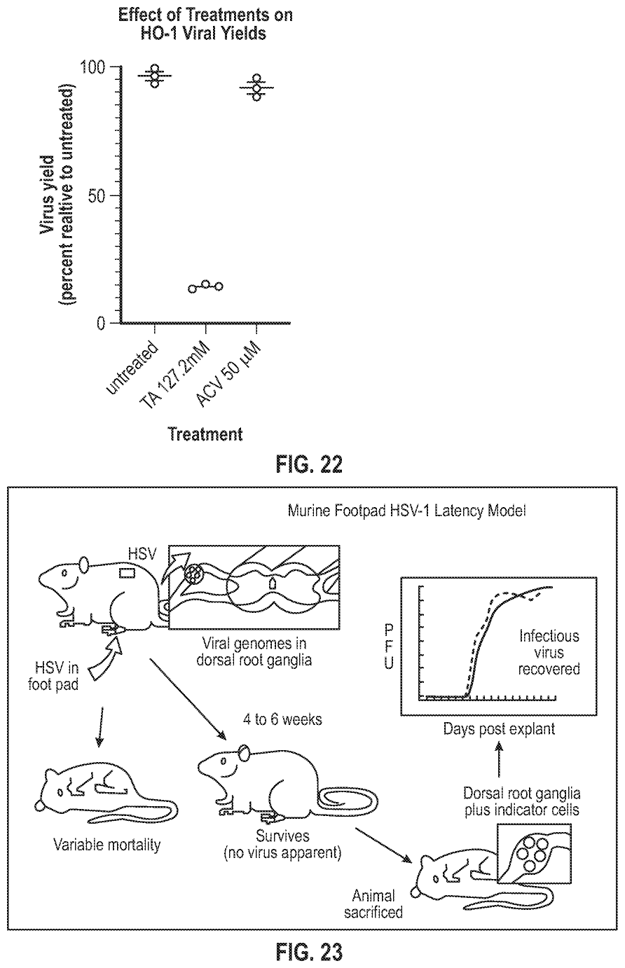

FIG. 22 illustrates effect of treatments on HO-1 viral yields. This data indicated that tranexamic acid is effective against a multiple drug resistant clinical isolate of HSV-1. This suggest tranexamic acid may provide a therapeutic option for drug-resistant HSV-1 infections of humans, such as, for example, HSV-1/HSV-2 skin lesions and HSV-1 stromal keratitis.

Efficacy of Tranexamic Acid Treatment against a Lethal HSV-1 Infection in a Mouse Footpad Model. Previous experiments demonstrated that tranexamic acid significantly reduces the production of infectious virus following HSV infections in vitro. This inhibition is dose-dependent and approaches the antiviral activity of acyclovir (FIG. 1). As such, tranexamic acid should reduce the infection and spread of HSV in a mouse model of HSV-1 infection.

The mouse footpad model of lethal HSV-1 infection is a well-established model of HSV-1 infection. It sensitively measures the ability of HSV-1 to replicate in the skin, invade into the nervous system, and spread. Mice are infected with HSV-1 on the plantar surfaces of their rear footpads. The virus replicates in the footpad epithelium, and then enters the nerve termini that innervate the skin. The virus travels up the sciatic nerve to the dorsal root ganglia neurons, where it will replicate and spread to the spinal cord. The virus will replicate in the spinal cord neurons and then spread to the brain. A proportion of the infected mice will succumb to HSV encephalitis in a virus strain and dose-dependent manner. Antivirals can be applied to the foot at the time of infection and assessed for their ability to interfere with viral replication and spread in vivo. FIG. 23 illustrates a murine footpad HSV-1 latency model.

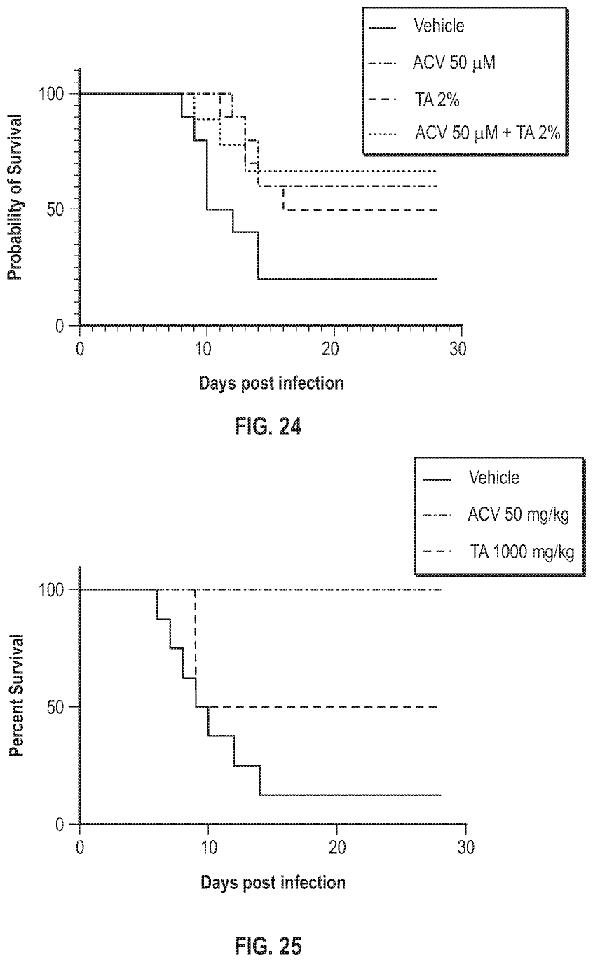

Experimental Design for Assessing the Efficacy of Tranexamic Acid in the Mouse Footpad Model of Lethal Infection. Mice were anesthetized with isoflurane and the plantar surface of both rear footpads of 4 to 6 week old female ND4 Swiss mice were pretreated with 10% saline (0.05 ml s.c.) to soften the cornified epithelium. 3 hr later the mice were anesthetized with a cocktail of xylazine, ketamine, and acepromazine (i.p.). The plantar surfaces of both rear footpads were lightly abraded with an emery board. The mouse footpads were treated with 25 μl of either vehicle, 2% tranexamic acid, or 50 μm acyclovir. There were 20 mice per treatment group. The mice were then infected with 25 μl of HSV-1 Strain 17syn+ (1000 pfu/mouse). The mice were monitored daily in a masked fashion for modified lethal endpoints (mice that showed bilateral hindlimb paralysis were not able to ambulate, or exhibited seizures were euthanized). FIG. 24 illustrates that tranexamic acid and acyclovir show synergy at reducing lethality of HSV-1 infection in the mouse footpad model.

Mice were anesthetized with isoflurane and the plantar surface of both rear footpads of 4 to 6 week old female ND4 Swiss mice were pre-treated with 10% saline (0.05 ml s.c.) to soften the cornified epithelium. 3 hr later the mice were anesthetized with a cocktail of xylazine, ketamine, and acepromazine (i.p.). The plantar surfaces of both rear footpads were lightly abraded with an emery board. The mice were then infected with 25 μl of HSV-1 Strain 17syn+ (1000 pfu/mouse). The mice were dosed with either vehicle, 50 mg/kg acyclovir, or 1000 mg/kg tranexamic acid once per day. The mice were monitored daily in a masked fashion for modified lethal endpoints (mice that showed bilateral hindlimb paralysis, were not able to ambulate or exhibited seizures were euthanized). FIG. 25 illustrates that tranexamic acid shows efficacy at reducing lethality of HSV-1 infection in the mouse footpad model.

The mouse footpad model demonstrated that tranexamic acid significantly reduces HSV-1 lethality in mice following footpad infection. The degree of efficacy is similar to that of acyclovir when administered topically. Furthermore, tranexamic acid and acyclovir appear to act synergistically to enhance mouse survival when applied topically. This data suggest that tranexamic acid may have significant potential as a safe and effective alternative to acyclovir in treating HSV-1 infections in humans.

Experimental Design for Assessing whether Tranexamic Acid can Reduce Acyclovir Escape Mutants. Combination viral therapies have proven effective for treating, for example, HIV/AIDS, and in preventing the development of drug-resistant mutants. Therefore tranexamic acid and acyclovir treatment may reduce the emergence of acyclovir resistant mutants. To study this, HSV Strain 17syn+ was serially passaged 10 times in the presence of either 50 μM acyclovir alone, or in combination with 2% tranexamic acid, in 60 mm dishes of confluent rabbit skin cells. Each passage was performed at an MOI of 0.01 pfu. At the end of the 10 passages, the stocks were plated on 100 mm dishes of rabbit skin cells in the presence of 100 μM acyclovir vs. no acyclovir. Acyclovir resistant mutants were counted and the percent of mutants determined. Table 1 illustrates tranexamic acid prevented the occurrence of acyclovir resistant mutants.

TABLE 1

| | Mock | Acyclovir | Acyclovir and |
|---|---|---|---|
| | Treated | Alone | Tranexamic Acid |
| Ratio of Acyclovir | None | 0.0044 | None Detected |
| Resistant (ACVr) Virus | Detected | (225/51,270) | |

Tranexamic acid significantly reduces the occurrence of acyclovir escape mutants. This data suggest that a treatment having a combination therapy of acyclovir and tranexamic acid may actually reduce the likelihood of acyclovir resistant strains of HSV emerging. This could be a significant advance given the increased incidence of acyclovir resistance.

Overall Findings and Results. As shown above, tranexamic acid significantly reduces HSV gene expression by 2 to 8 fold. Tranexamic acid's effect on transcription of HSV immediate early genes indicates that it acts very early in the infection cycle. In contrast, acyclovir acts later in infection by inhibiting DNA replication. This data point to a novel mechanism of blocking viral transcription that occurs very early after infection, a property that may have significant therapeutic advantages. Tranexamic acid may be acting to either interfere with transcription of viral genes directly, or indirectly through the activation of intrinsic antiviral responses.

As demonstrated above, tranexamic acid exhibits significant antiviral activity against HSV-1 both in vitro and in vivo. The antiviral action of tranexamic acid is manifested as interference with HSV-1 lytic gene transcription, and this occurs very early after infection. Additionally, tranexamic acid exhibits a synergistic enhancement of acyclovir treatment both in vitro and in vivo. Tranexamic acid is especially effective at blocking HSV-1 infection in vivo following topical application in mice. Finally, tranexamic acid, when given in combination with acyclovir, reduces the occurrence of acyclovir resistant mutants below the level of detection in vitro. Therefore, tranexamic acid may have significant potential as both an alternative therapy to acyclovir for treating acyclovir resistant strain of HSV, as well as in an efficacious combination therapy that can prevent the development of acyclovir resistance.

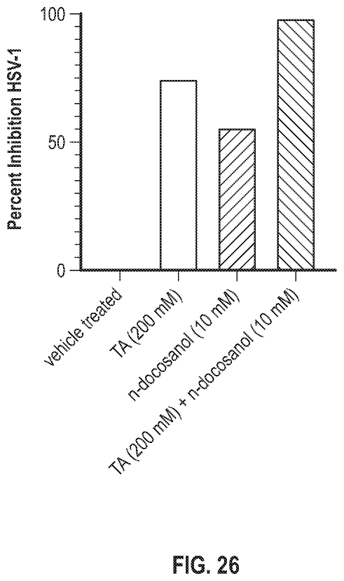

Tranexamic Acid and Docosanol. Confluent monolayers of rabbit skin cells were pre-treated with either tranexamic acid, n-docosanol, tranexamic acid and n-docosanol, or vehicle for 12 hr. Cells were then infected with HSV-1 at an MOI of 1. 12 hours later the cells were harvested, frozen, subjected to 2 freeze-thaw cycles, and then titrated for infectious viruses by standard plaque assay. Results, illustrated in FIG. 26, are presented as percent inhibition of HSV-1 yields (relative to vehicle treated control). FIG. 26 highlights the synergistic properties of tranexamic acid and docosanol.

It is envisioned that the synergistic combinations herein can allow for lower doses of either tranexamic acid, acyclovir, docosanol, or other antiviral with a similar mechanism of action to that of acyclovir or docosanol, to be given in combination. Further, it is envisioned that the use of tranexamic acid and acyclovir, docosanol, or other antiviral with a similar mechanism of action to that of acyclovir or docosanol, in a synergistic combination can reduce the occurrence of drug-resistant strains or mutations of HSV-1 and HSV-2, especially in immunocompromised hosts.

Treatment and Prophylactic Use. In addition to the data described above, various treatment and prophylactic use studies have been conducted and recorded for human subjects. For example, treatment activity is illustrated via a 54 year old female subject with a history of recurrent outbreaks of cold sores on or near the lips. In this instance, the subject noticed the first signs of the outbreak, in this case, a red blemish with small white spots surrounding it along with associated tingling, pain, and sensitivity, and immediately applied a small amount, approximately 0.25 mL of an aqueous solution of 5% (w/v) tranexamic acid to the area via a simple swab. This practice was repeated 5 times over the course of 36 hours and surprisingly within that 36 hour period the cold sore had healed to the point that only a small red spot was visible, which was totally resolved shortly thereafter. This activity was a marked improvement in the typical duration of the subject's outbreaks, which usually lasted approximately 14 days, even when using a topical antiviral treatment, such as ABREVA®. While a 5% (w/v) concentration of tranexamic acid proved to be effective, a range of concentrations and total dosages delivered, such as, 0.5 to 30% (w/v) delivered in increments of 0.25 to 5 mL over the course of 1 to 14 days, for example, can prove beneficial as well.

In addition to the treatment study mentioned above, further studies have been conducted in which three human subjects with an experience of recurring cold sores, generally lasting for about 2 weeks, applied 3% (w/v) tranexamic acid topically several times a day upon detection of the beginning of an outbreak, and the symptoms of the outbreak resolved within 48 hours. A fourth subject with an experience of recurring cold sores, who usually applied ABREVA® for several days just to avoid a larger outbreak, applied 10% (w/v) tranexamic acid topically a single time upon feeling the onset of an outbreak and noticing a very small blister, and the following morning the blister was gone and the outbreak had ceased. Furthermore, one of the subjects applied 3 to 10% (w/v) tranexamic acid to their face daily for approximately one year, and experienced only one cold sore outbreak during that year, rather than their usual experience of 3 to 5 outbreaks.

Moreover, there have been three instances where individuals who have applied a solution of 3% (w/v) tranexamic acid to their nasal passages and throat every 6 to 8 hours when they sensed the symptoms of a cold or influenza infection, and the symptoms resolved within 36 to 48 hours, rather than the usual duration of around 2 weeks.

In view of the data of tranexamic acid in both treatment and prophylactic use, tranexamic acid show effects of enhancing immune response in subjects. This data, in conjunction with data discussed in detail above, indicates tranexamic acid shows secondary antiviral effects due to its immune system enhancement. As such, in certain embodiments, tranexamic acid can be administered in combination with various other pharmaceutical agents, such as, but not limited to, vaccines, in order to enhance immune response in a patient. Accordingly, in some embodiments, tranexamic acid can be administered as an adjuvant.

In view of the foregoing, an embodiment of the present disclosure is directed to the application of a synergistic combination of synthetic lysine analogs, derivatives, mimetics, or prodrugs in combination with a pharmaceutical agent. In some embodiments, the pharmaceutical agent is an antiviral agent to provide for viral inhibition by utilizing the pharmacologic activity of the synergistic combination. In some embodiments, the synthetic lysine analogs, derivatives, mimetics, or prodrugs can be tranexamic acid. In some embodiments, the synthetic lysine analogs, derivatives, mimetics, or prodrugs can be epsilon-aminocaproic acid (EACA) or AZD 6564. In some embodiments, the pharmaceutical agent can be acyclovir. In some embodiments, the pharmaceutical agent can be famciclovir, ganciclovir, penciclovir, valaciclovir, or valganciclovir. In some embodiments, the pharmaceutical agent can be docosanol. In some embodiments, the pharmaceutical agent can include, without limitation, an antimicrobial agent, an anticancer agent, a genetic therapy agent, an immune-enhancing agent, a hormonal therapy agent, an antiviral antibody, and combinations thereof.

In some embodiments, the pharmaceutical agent can be a nucleoside analogue, including, without limitation, deoxyadenosine analogues, adenosine analogues, deoxycytidine analogues, guanosine and deoxyguanosine analogues, thymidine and deoxythymidine analogues, deoxyuridine analogues, and combinations thereof. In some embodiments, the pharmaceutical agent can include, without limitation, didanosine, vidarabine, cytarabine, gemcitabine, emtricitabine, lamivudine, zalcitabine, abacavir, aciclovir, entecavir, stavudine, telbivudine, zidovudine, idoxuridine, trifluridine, and combinations thereof. In some embodiments, the pharmaceutical agent can include, without limitation, nucleobase analogues, nucleotide analogues, and combinations thereof.

In some embodiments, the pharmaceutical agent can be multiclass combination drugs, including, but not limited to, abacavir/dolutegravir/lamivudine, darunavir/cobicistat/emtricitabine/tenofovir alafenamide, dolutegravir/rilpivirine, elvitegravir/cobicistat/emtricitabine/tenofovir disoproxil fumarate, elvitegravir/cobicistat/emtricitabine/tenofovir alafenamide, efavirenz/emtricitabine/tenofovir disoproxil fumarate, emtricitabine/rilpivirine/tenofovir disoproxil fumarate, emtricitabine/rilpivirine/tenofovir alafenamide, bictegravir/emtricitabine/tenofovir alafenamide, and combinations thereof. In some embodiments, the pharmaceutical agent can be integrase inhibitors including, but not limited to, dolutegravir, elvitegravir, raltegravir, raltegravir extended-release, and combinations thereof.

In some embodiments, the pharmaceutical agent can be nucleoside/nucleotide reverse transcriptase inhibitors (NRTIs) including, but not limited to, abacavir, abacavir/lamivudine, abacavir/lamivudine/zidovudine, lamivudine/zidovudine, lamivudine, zidovudine, emtricitabine/tenofovir disoproxil fumarate, emtricitabine, tenofovir disoproxil fumarate, emtricitabine/tenofovir alafenamide, didanosine, didanosine extended-release, stavudine, and combinations thereof. In some embodiments, the pharmaceutical agent can be non-nucleoside reverse transcriptase inhibitors (NNRTIs) including, but not limited to, efavirenz, etravirine, nevirapine, nevirapine extended-release, rilpivirine, delavirdine mesylate, and combinations thereof.

In some embodiments, the pharmaceutical agent can be protease inhibitors including, but not limited to, atazanavir/cobicistat, darunavir/cobicistat, lopinavir/ritonavir, ritonavir, atazanavir, darunavir, fosamprenavir, tipranavir, nelfinavir, indinavir, saquinavir, and combinations thereof. In some embodiments, the pharmaceutical agent can be entry inhibitors (including fusion inhibitors) including, but not limited to, enfuvirtide. In some embodiments, the pharmaceutical agent can be chemokine co-receptor antagonists (CCR5 antagonists) including, but not limited to, maraviroc. In some embodiments, the pharmaceutical agent can be cytochrome P4503A (CYP3A) inhibitors including, but not limited to, cobicistat, ritonavir, and combinations thereof. In some embodiments, the pharmaceutical agent can include immune-based therapies. In some embodiments, the pharmaceutical agent is an adjuvant treatment or therapy agent. In some embodiments, the pharmaceutical agent is an antiviral antibody.

In some embodiments, the pharmaceutical agent can be, for example, oseltamivir (used as an anti-influenza therapy), which inhibits the neuraminidase enzyme that allows newly formed viruses to exit the host cell, the very last stage of viral replication and spreading. Similarly, in some embodiments, the pharmaceutical agent can be baloxavir marboxil, which inhibits polymerase acidic endonuclease, an enzyme that allows for replication of the viral DNA, which is in the middle stage of viral replication and is different from the mechanism of action for tranexamic acid.

In some embodiments, the synergistic combination may be in the form of a simple aqueous solution, a solution with inert excipients, or combined with vehicles such as a gel, cream, or lotion, which may optionally contain other treatment ingredients. An additional embodiment to improve handling or treatment delivery, such as via viscous solutions or solutions designed to delay, slow, or predictably deliver the synergistic combination are also envisioned. In some embodiments, the synergistic combination may be formulated to be administered in a nasal passage. In some embodiments, the synergistic combination may be formulated to be administered in an upper airway.

In some embodiments, the synergistic combination can be directly administered to an area of skin that is showing signs of a viral outbreak or affected by some other disease or condition. In some embodiments, the synergistic combination can be in a topical form. In some embodiments, the synergistic combination can be in pill, tablet, or capsule form. In some embodiments, the synergistic combination can be delivered systemically. In some embodiments, the synergistic combination can be easy to apply, for example, by being adaptable to the affected area. In some embodiments, the synergistic combination can utilize the activity of the synergistic combination at the first sign of a viral outbreak, such as HSV-1, to reduce the severity and duration of the outbreak and promote rapid healing. In some embodiments, the synergistic combination may be applied on a frequent, such as, for example, a daily basis, to avoid an outbreak or an occurrence of a disease.

In some embodiments, the synergistic compound can suppress future viral outbreaks. In some embodiments, the synergistic compound inhibits viral development, for example, but not limited to, inhibiting the viral development of HIV. In some embodiments, usage of the synergistic combination provides for viral latency. For example, in HSV-1, usage of the synergistic combination can reduce the number of viral outbreaks. In some embodiments, usage of the synergistic combination can allow for outbreaks to be significantly reduced or eliminated.

In some embodiments, treatment can be practiced with systemic administration of the synergistic combination, but can additionally be applied in a topical form in effective concentrations and regimens in order to provide rapid activity and benefits. In some embodiments, the concentration of the synergistic combination can be, for example, up to 60% (w/v) concentration of the synthetic lysine analog, derivative, mimetic, or prodrug, and up to the regularly prescribed amount of the pharmaceutical agent. In some embodiments, the synergistic combination is in topical form and the concentration of the synergistic combination is up to 20% (w/v) of the synthetic lysine analog, derivative, mimetic, or prodrug, and up to a standard prescribed dose of the pharmaceutical agent.

Moreover, an embodiment of the present disclosure is directed to the application of a synergistic combination to provide for the enhancement of the efficacy of a pharmaceutical agent or a lysing analog, derivative, mimetic, or prodrug, by utilizing the pharmacologic activity of the synergistic combination. The synergistic combination may be a simple aqueous solution, a solution with inert excipients, or combined with vehicles such as a gel, cream, or lotion which may optionally contain other treatment ingredients. In some embodiments, the synergistic combination is formulated to be administered in a nasal passage or an upper airway of a subject.

Additional embodiments to improve handling or prophylactic delivery, such as, for example, viscous solutions or solutions designed to delay or predictably deliver the synergistic combination are also envisioned. The synergistic combination can be directly administered to an area of skin and can be easy to apply via adaptability to the desired area of application. In some embodiments, the synergistic combination can be in a topical form. In some embodiments, the synergistic combination can be in pill, tablet, or capsule form. In some embodiments, the concentration of the synergistic combination can be, for example, up to 60% (w/v) concentration of the synthetic lysine analog, derivative, mimetic, or prodrug, and up to a standard prescribed dose of the pharmaceutical agent. In some embodiments, the synergistic combination is in topical form and the concentration of the synergistic combination is up to 20% (w/v) of the synthetic lysine analog, derivative, mimetic, or prodrug, and up to a standard prescribed dose of the pharmaceutical agent.

In some embodiments, the synergistic combination can be used for the prevention or treatment of infections and diseases caused by, for example, viruses including, but not limited to, HIV, the common cold and influenza viruses, or other transient viruses (e.g., corona viruses), for example in persons at increased risk of exposure, or who have been exposed to infection by such viruses but do not yet exhibit symptoms of infection, or persons for whom infection by such viruses could represent a life-threatening event. As several of these viruses attach to the back of the throat and nasal passages, in some embodiments, the synergistic combination can be formulated into sprays, mists, aerosols, mouth washes, or solutions to be swabbed, that can be applied to mouth, nose, or throat areas, including, for example, the nasal passages or the upper airway. In some embodiments, the synergistic combination can be used for the prevention or treatment of infections and diseases caused by transient viruses, such as, for example, corona viruses,

In some embodiments, the synergistic combination, presented herein, can be utilized for the prevention of viral outbreaks or suppression of the development of viral infections, or the prevention or suppression of other diseases or conditions, and can be administered via enteral and parenteral methods, for example, pills, tablets, capsules, or injections. In some embodiments, the synergistic combination can be administered via an injected or implanted liposomal delivery depot for long-term administration. In some embodiments, the synergistic combination can be in the form of a transdermal patch that administers the drug via skin contact.

In some embodiments, the present disclosure relates to a synthetic lysine analog, derivative, mimetic, or prodrug and a pharmaceutical agent, and method of use thereof, such that the synthetic lysine analog, derivative, mimetic, or prodrug and the pharmaceutical agent form a synergistic combination to enhance the efficacy of the pharmaceutical agent or the synthetic lysine analog, derivative, mimetic, or prodrug. In some embodiments, the synergistic combination is in a solution. In some embodiments, the solution is formulated to be administered in a nasal passage. In some embodiments, the solution is formulated to be administered in an upper airway. In some embodiments, the solution is formulated as a spray, mist, aerosol, or mouthwash. In some embodiments, the solution is formulated to be applied as part of a vehicle which adapts to human skin. In some embodiments, the solution is formulated to be administered intravenously. In some embodiments, the solution is formulated to be applied via a vehicle that allows the synergistic combination to be delivered in a time-released fashion.

In some embodiments, the synergistic combination has a concentration of about 1% to about 60% by weight of the synthetic lysine analog, derivative, mimetic, or prodrug and about one-quarter to a standard dose of the pharmaceutical agent. In some embodiments, the synergistic combination has a concentration of about 1% to about 60% by weight of the synthetic lysine analog, derivative, mimetic, or prodrug and about one-half to a standard dose of the pharmaceutical agent. In some embodiments, the synergistic combination is formulated to be delivered orally. In some embodiments, the synergistic combination is formulated to be delivered in a time-released fashion.

In some embodiments, the synergistic combinations herein can allow for lower doses of the lysine analog, derivative, mimetic, or prodrug, or the pharmaceutical agent to be given in combination. In some embodiments, the synergistic combinations herein can allow for lower doses of either tranexamic acid or the pharmaceutical agent, for example, acyclovir to be given in combination. In some embodiments, the use of the lysine analog, derivative, mimetic, or prodrug and the pharmaceutical agent can reduce the occurrence of drug-resistant strains, mutations, or the like of various diseases. In some embodiments, the use of tranexamic acid and the pharmaceutical agent (e.g., acyclovir) in the synergistic combination can reduce the occurrence of drug-resistant strains or mutations of viruses. In some embodiments, the synergistic combination can reduce the occurrence of drug-resistant strains or mutations of HSV-1 and HSV-2. In some embodiments, the reduction of the occurrence of drug-resistant strains or mutations of HSV-1 and HSV-2 is in immunocompromised hosts.

In some embodiments, the synergistic combination can be in the form of a same solution, tablet, or capsule, such that the synergistic combination can be administered in a same medium (e.g., a tablet). In some embodiments, the synergistic combination can be a combination of two separate mediums. For instance, in some embodiments, the synthetic lysine analog, derivative, mimetic, or prodrug can be in the form of a first medium and the pharmaceutical agent can be in the form of a second medium. In embodiments where the synthetic lysine analog, derivative, mimetic, or prodrug and the pharmaceutical agent are in separate mediums, the synergistic combination can be in the form of a kit. In some embodiments, the kit includes the synthetic lysine analog, derivative, mimetic, or prodrug in a first medium. In some embodiments, the first medium is a solution, tablet, or capsule. In some embodiments, the kit includes the pharmaceutical agent in a second medium. In some embodiments, the second medium is a solution, tablet, or capsule. In embodiments where the synergistic combination is in the form of a kit, the kit can include various combinations of mediums. For example, in some embodiments, the kit can include a solution-based lysine analog, derivative, mimetic, or prodrug and a capsule-based pharmaceutical agent. In some embodiments, each component of the kit can be administered at different times to utilize peak drug metabolism (e.g., pharmacokinetics).

In some embodiments, the kit can include, without limitation, a synthetic lysine analog, derivative, mimetic, or prodrug and a pharmaceutical agent, where the synthetic lysine analog, derivative, mimetic, or prodrug and the antiviral agent form a synergistic combination. In some embodiments, the synthetic lysine analog, derivative, mimetic, or prodrug is in a first medium and the pharmaceutical agent is in a second medium. In some embodiments, at least one of the first medium and the second medium is a pill, tablet, or capsule. In some embodiments, at least one of the first medium and the second medium is a solution.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1-44. (canceled)

45. A kit to enhance efficacy of a pharmaceutical agent, the kit comprising:
a synthetic lysine analog, derivative, mimetic, or prodrug; and
the pharmaceutical agent, wherein the synthetic lysine analog, derivative, mimetic, or prodrug and the antiviral agent form a synergistic combination; and
wherein the pharmaceutical agent is selected from the group consisting of acyclovir, famciclovir, ganciclovir, penciclovir, valaciclovir, or valganciclovir, deoxyadenosine analogues, adenosine analogues, deoxycytidine analogues, guanosine and deoxyguanosine analogues, thymidine and deoxythymidine analogues, deoxyuridine analogues, didanosine, vidarabine, cytarabine, gemcitabine, emtricitabine, lamivudine, zalcitabine, abacavir, aciclovir, entecavir, stavudine, telbivudine, zidovudine, idoxuridine, trifluridine, oseltamivir, baloxavir marboxil, docosanol, and combinations thereof.

46. The kit of claim 45, wherein the synthetic lysine analog, derivative, mimetic, or prodrug is selected from the group consisting of tranexamic acid, epsilon-aminocaproic acid (EACA), and AZD 6564.

47. The kit of claim 46, wherein the synthetic lysine analog, derivative, mimetic, or prodrug is tranexamic acid.

48-49. (canceled)

50. The kit of claim 45, wherein the pharmaceutical agent is acyclovir.

51. The kit of claim 45, wherein the pharmaceutical agent is docosanol.

52. The kit of claim 45, wherein the pharmaceutical agent is an adjuvant treatment or therapy agent.

53. The kit of claim 45, wherein the synthetic lysine analog, derivative, mimetic, or prodrug is in a first medium and the pharmaceutical agent is in a second medium.

54. The kit of claim 52, wherein at least one of the first medium and the second medium is a pill, tablet, or capsule.

55. The kit of claim 52, wherein at least one of the first medium and the second medium is a solution.

56. The kit of claim 55, wherein the solution has a concentration of 0.5 to 30% by weight of the synthetic lysine analog, derivative, mimetic, or prodrug.

57. The kit of claim 55, wherein the solution is formulated to be administered in a nasal passage or an upper airway.

58. The kit of claim 55, wherein the solution is formulated to be applied as part of a vehicle which adapts to human skin.

59. The kit of claim 58, wherein the vehicle is selected from the group consisting of a gel, a lotion, and a cream.

60. The kit of claim 45, wherein the synergistic combination has a concentration of 1% to 60% by weight of the synthetic lysine analog, derivative, mimetic, or prodrug and one-eighth up to a standard dose or more of the pharmaceutical agent.

61. The kit of claim 45, wherein the synergistic combination is administered at least once per day.

62. The kit of claim 45, wherein the synthetic lysine analog, derivative, mimetic, or prodrug comprises 3 to 10% (w/v) tranexamic acid.

63. The kit of claim 45, wherein the synthetic lysine analog, derivative, mimetic, or prodrug comprises 5% (w/v) tranexamic acid.

64. The kit of claim 45, wherein the synthetic lysine analog, derivative, mimetic, or prodrug comprises 10% (w/v) tranexamic acid.

65. The kit of claim 45, wherein the synthetic lysine analog, derivative, mimetic, or prodrug is in a first medium and the pharmaceutical agent is in a second medium, the first medium comprising 3 to 10% (w/v) tranexamic acid and the second medium comprising at least one of acyclovir or docosanol.

66. The kit of claim 45, wherein the synthetic lysine analog, derivative, mimetic, or prodrug is in a first medium and the pharmaceutical agent is in a second medium, the first medium comprising 5% (w/v) tranexamic acid and the second medium comprising docosanol.
